(19) United States
(12) Patent Application Publication
(10) Pub. No.: US 2025/0204795 A1
(43) Pub. Date: Jun. 26, 2025

(54) ANIMAL SURGICAL MONITOR SYSTEM WITH INTEGRATED ECG AND PULSE OXIMETER

(71) Applicant: Sridhar Madala, Houston, TX (US)

(72) Inventor: Sridhar Madala, Houston, TX (US)

(21) Appl. No.: 18/953,506

(22) Filed: Nov. 20, 2024

Related U.S. Application Data
(60) Provisional application No. 63/613,031, filed on Dec. 20, 2023.

(51) Int. Cl. A61B 5/0205; A61B 5/024; A61B 5/0245; A61B 5/1455

(52) U.S. Cl. CPC A61B 5/02055; A61B 5/02427; A61B 5/0245; A61B 5/14552; A61B 2503/42

(57) ABSTRACT

An improved sensor assembly and sensing system for measuring physiologic parameters of a test animal subject. In various embodiments the present subject matter provides an integrated electrocardiogram (ECG) electrode and plethysmographic sensor for measuring ECG and pulse oximetry (SPO2) data from an animal subject. The present sensor assembly and system provide measurements of heart rate, heart rate variability (HRV), cardiac activity (including abnormal cardiac activity such as arrythmia, fibrillation, tachycardia, premature ventricular contractions, etc.), respiration rate (RespR), correlated (uncalibrated) blood pressure (systolic, diastolic, and pulse pressure), and pulse oximetry (SPO2). In various examples, the integrated sensor is configured in the shape of the test subject and to provide an outline of electrodes and pulse oximetry sensors that are configured at a predefined locations to facilitate sensing. The integrated sensor may be adjustably heated. Wired and wireless variations may be employed for all communications between the integrated sensor and a computing device, such as a table, laptop, desktop, smartphone, or other computer or computing system.

### PRIORITY APPLICATIONS

This application claims the benefit of priority to U.S. Provisional Patent Application Ser. No. 63,613,031, filed Dec. 20, 2023, the content of which is incorporated herein by reference in its entirety.

### TECHNICAL FIELD

The present disclosure pertains to methods and apparatus for an animal surgical monitor system, and more particularly to method and apparatus for a small animal surgical monitor that provides a number of physiological measurements.

### BACKGROUND

Laboratory research frequently involves the monitoring of physiological parameters of subject animals for physiologic testing, such as rodents. The physiological monitoring of rodents, such as mice, may pose some challenges because their anatomy is relatively small and they typically have substantially higher heart rates, such as in the range of 200-900 beats per minute.

Workers in the field have proposed different monitors to collect information from the subject animal. For example, U.S. Pat. No. 8,005,624 to Starr Life Sciences Corp. (“the '624 patent”) demonstrates an incandescent lamp or light emitting diode (LED) that illuminates an appendage of the subject animal having a sufficient amount of blood to serve as a plethysmograph. The '624 patent proposes a method of supplying rodents with pre-installed or embedded physiologic sensors to medical researchers to address the issues of sensing limbs of small animals, such as mice. It discusses pre-installed (for example, subcutaneous) sensors with wireless hardware that can be read by a computer which acts as a wireless monitoring station. The '624 patent also proposes an external pulse oximeter in a spring biased clip that is designed to be used on a paw of the rodent. However, clips fall off and can give inconsistent results for the tested animal. The use of pre-installed or implanted sensors can be expensive and may impact the overall health of the test subject regardless of the testing. Also, the spring biased clip may be hard to maintain in place and can injure the animal or slip off, losing the ability to measure and record the pulse oximetry data from the subject animal.

When rodents are anesthetized either for non-invasive studies or for surgery, their temperature regulating mechanisms may not be adequate to maintain their physiological body temperature. This can lead to a rapid drop in body temperature in a few minutes and can have adverse results. The conventional solution to this problem is a cumbersome pad with circulating hot water or an overhead heat lamp that can cause electrical interference with the ECG monitoring equipment.

There is a need in the art for a better sensor and system for sensing physiologic parameters. Such a sensor should be used with minimal impact to the test subject and such a system should provide reliable sensing of many physiological parameters of the test subject.

### SUMMARY

Previous approaches have relied on external clips or sensors requiring implantation, which may be expensive and may injure the animal and affect its health. The present animal surgical monitor system with its improved sensor pad avoids these downsides and provides reliable animal testing data.

The present subject matter provides, among other things, an improved integrated sensor assembly and monitoring system for measuring physiological parameters of small animal test subjects. The system utilizes an integrated pad with electrocardiogram electrodes and a pulse oximetry sensor configured to match the shape of the animal. This allows for placement of the animal on the pad to simultaneously obtain measurements of heart rate, heart rate variability, cardiac activity, respiration rate, blood pressure, and blood oxygen saturation. The system processes the high frequency signals from small animals to provide accurate measurements.

In various embodiments, the present system provides an improved sensor assembly and sensing system for measuring physiologic parameters of a test animal subject. In various embodiments the present subject matter provides an integrated electrocardiogram (ECG) electrode and plethysmographic sensor for measuring ECG and pulse oximetry (SpO₂) data from an animal subject. The present sensor assembly and system provide measurements of heart rate, heart rate variability (HRV), cardiac activity (including abnormal cardiac activity such as arrythmia, fibrillation, tachycardia, premature ventricular contractions, etc.), respiration rate (RespR), correlated (uncalibrated) blood pressure (systolic, diastolic, and pulse pressure), and pulse oximetry (SpO₂). In various embodiments the sensor assembly includes inputs for temperature sensing. In various embodiments the sensor assembly includes inputs for blood pressure sensing. In various embodiments, the sensor assembly provides adjustable levels of heat for the subject animal. In various embodiments, the system processes high frequency signals from small animals to provide accurate measurements. In various embodiments, the sensor is configured in the shape of the test subject and to provide an outline of electrodes and pulse oximetry sensors that are configured at a predefined locations to facilitate sensing. For example, in the case of rodent sensing, such as a mouse, the sensor arrangement allows for the placement of the mouse on a preconfigured electrode and sensing pad for measurement of a number of physiological parameters. In various embodiments, the present system can provide an integrated sensor that may be used with rodents generally, providing separate sensors for mouse and rat subjects, which are typically different sizes. In various embodiments, the system can be programmed to determine which sensors to use to best sense the parameters of the mouse, rat, or other subject. Those of skill in the art will appreciate that other shapes and test subjects and sensor configurations may be used in various combinations and subcombinations without departing from the scope of the present subject matter.

This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is a diagram of various components of an animal surgical monitor system according to one embodiment of the present subject matter.

FIG. 2 shows inputs and outputs and sensors of an integrated sensor for the animal surgical monitor system according to one embodiment of the present subject matter.

FIG. 3 shows an example profile of an integrated sensor without an anesthesia mounting bracket according to one embodiment of the present subject matter.

FIG. 4 is an end perspective view of the integrated sensor of FIG. 3.

FIGS. 5 and 6 are right and left side views of the integrated sensor of FIG. 3.

FIGS. 7 and 8 are bottom and top views of the integrated sensor of FIG. 3.

FIG. 9 is a back side view of the integrated sensor of FIG. 3.

FIG. 10 is a side perspective view of the integrated sensor of FIG. 3.

FIGS. 11A and 11B demonstrate prone animal positioning on the sensor in the rostral and caudal positions, respectively, according to various embodiments of the present subject matter.

FIGS. 12A and 12B demonstrate supine animal positioning on the sensor in the rostral and caudal positions, respectively, according to various embodiments of the present subject matter.

FIGS. 13A and 13B show the active optical sensor for supine-rostral positioning for sensing mouse and rat pulse oximetry, respectively, according to one embodiment of the present subject matter.

FIG. 14 is an example software interface for programming the pulse oximeter sensor, according to one embodiment of the present subject matter.

FIG. 15 shows an example of programming the pulse oximeter sensor for mouse configuration sensing, according to one embodiment of the present subject matter.

FIG. 16 shows an example of programming the pulse oximeter sensor for rat configuration sensing, according to one embodiment of the present subject matter.

FIG. 17 shows an example of programming filters for the pulse oximetry sensor, according to one embodiment of the present subject matter.

FIG. 18 shows an example of programming vertical scaling for the pulse oximetry sensor, according to one embodiment of the present subject matter.

FIG. 19 shows an example of programming advanced features for the pulse oximetry sensor, according to one embodiment of the present subject matter.

FIG. 20 is a block diagram of a system in the example form of a computing system within which a set of instructions may be executed, for causing the machine to perform any one or more of the methodologies discussed herein.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The scope of the present invention is defined by the appended claims, along with the full scope of legal equivalents to which such claims are entitled.

In various embodiments, the present disclosure provides an improved sensor assembly and sensing system for measuring physiologic parameters of a test animal subject. In various embodiments the system provides an integrated electrocardiogram (ECG) electrode and plethysmographic sensor for measuring ECG and pulse oximetry (SpO₂) data from an animal subject. The present sensor assembly and system provide measurements of heart rate, heart rate variability (HRV), cardiac activity (including abnormal cardiac activity such as arrythmia, fibrillation, tachycardia, premature ventricular contractions, etc.), respiration rate (RespR), correlated (uncalibrated) blood pressure (systolic, diastolic, and pulse pressure), and pulse oximetry (SpO₂).

In various embodiments, the sensor is configured in the shape of the test subject and to provide an outline of electrodes and pulse oximetry sensors that are configured at a predefined locations to facilitate sensing. For example, in the case of rodent sensing, such as a mouse, the sensor arrangement allows for the placement of the mouse on a preconfigured electrode and sensing pad for measurement of a number of physiological parameters. In various embodiments, the present system can provide an integrated sensor that may be used with rodents generally, providing separate sensors for mouse and rat subjects, which are typically different sizes. In various embodiments, the system can be programmed to determine which sensors to use to best sense the parameters of the mouse, rat, or other subject. Those of skill in the art will appreciate that other shapes and test subjects and sensor configurations may be used in various combinations and subcombinations without departing from the scope of the present subject matter.

The system executes software to configure and monitor the integrated sensor. In various embodiments the sensor of the system provides a heated surgical platform that is used to acquire and process and display ECG, respiration, pulse plethysmograph, and blood pressure waveforms, as well as heart rate, breath rate, core temperature, SpO₂, and systolic & diastolic pressures from rodents such as mice and rats. The unique integrated operating platform incorporates ultra-low noise, high-resolution ECG electronics and intelligent zone heating with a durable surgical stainless-steel top. The included touchscreen tablet display communicates wirelessly with the platform and ergonomically presents waveform and numeric data in an intuitive and easily readable format, enabling the quick recording and exporting of the acquired data via Analog Output channels that are compatible with most data acquisition systems.

Specifically, the system adapts and modifies the signal processing to accommodate the higher heart rates seen in small animals such as rodents. This includes settings for high pass, low pass, and notch filters optimized for the frequency ranges expected. For example, the high pass filter removes baseline drift, while the low pass filter eliminates high frequency noise. The notch filter suppresses any 50 Hz or 60 Hz power line interference.

Additionally, the calibration equation incorporated in the processing algorithm is tailored to the wavelengths and optical properties of rodent blood rather than human blood. This allows more accurate calculation of oxygen saturation from the red and infrared plethysmography signals.

The target ranges for the control loop that adjusts the LED brightness are also set based on the small amplitude signals expected from small animals. This allows the system to optimize the plethysmography waveforms for the best pulse oximetry data.

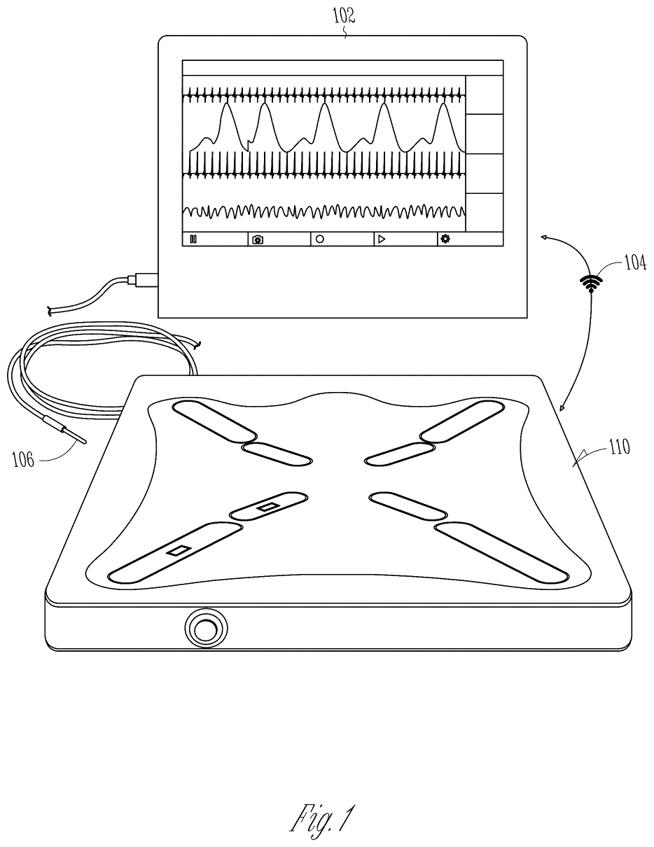

FIG. 1 is a diagram of various components of an animal surgical monitor system according to one embodiment of the present subject matter. A computing device 102 communicates with an integrated sensor 110 over a wired or wireless connection 104 to configure and control the integrated sensor 110 and to receive physiological measurements from an animal placed on the integrated sensor 110. In various embodiments the system accommodates additional sensors, such as temperature probe 106 which may be used to record the subject animal's temperature. Other sensors may be combined without departing from the scope of the present subject matter. In various embodiments the computing device is a tablet PC, a laptop PC, a desktop PC, a smartphone, or any other digital device. In devices that have a display the system may output real time and/or post-processed data sensed from the subject on the integrated sensor 110. The wireless connections may be made between the integrated sensor 110 and the computing device 102 via Wifi, Bluetooth, Zigbee, LoRa, NFC, WiMAX, 3G, 4G, 5G, LTE, 915 MHz, ISM frequency band, or other wireless interfaces, including those not yet invented. The wired connections can be through any wired interface. Communications can be wireless between the integrated sensor and other sensor or other digital devices without departing from the scope of the present subject matter.

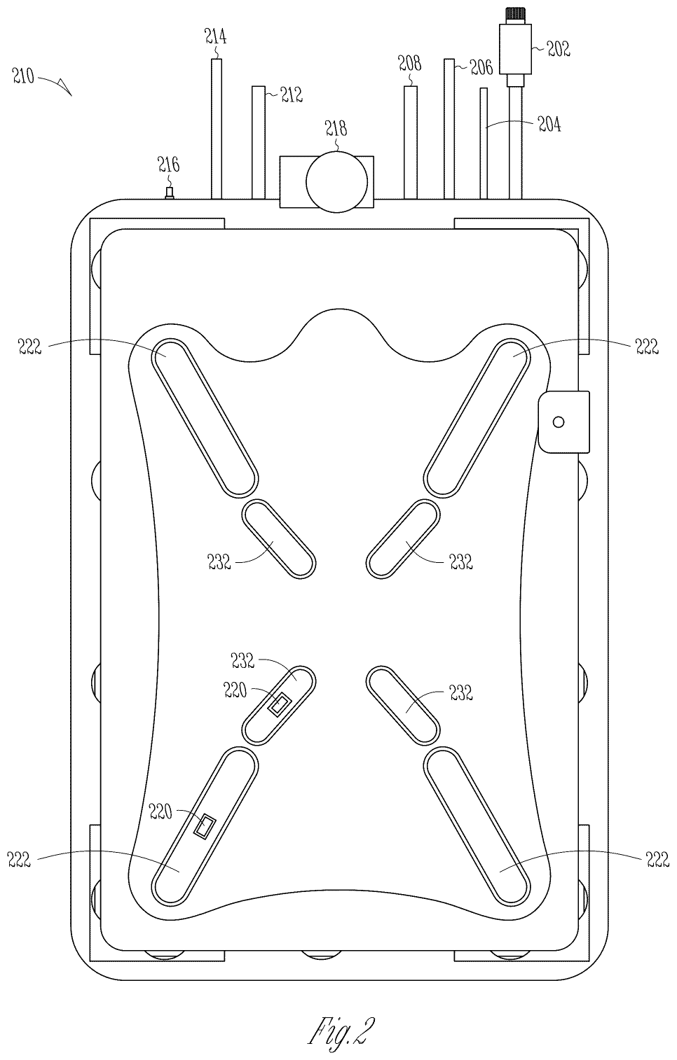

FIG. 2 shows inputs and outputs and sensors of an integrated sensor for the animal surgical monitor system according to one embodiment of the present subject matter. The example set forth demonstrates the sensor for rodents, but persons of skill in the art will appreciate that the sensor may be adapted for a variety of animal applications. In the example of FIG. 2, Mouse ECG electrodes 232 provide electrical sensing of the four limbs of a mouse, and a slightly larger electrode size and electrode spacing is provided for rat ECG electrodes 222. At least one of the electrode pads is designed to accommodate an optical plethysmographic sensor to provide pulse oximetry (SpO₂) data from an animal subject, for example, optical sensor 220 for the mouse application and optical sensor 230 for the rat application. It is understood that these sensors 220 and 230 may be positioned in one or more of the ECG electrode positions to facilitate pulse oximetry sensing. In various embodiments a special purpose integrated circuit for SpO₂ measurement is employed, such as an Analog Devices MAXM86161 Integrated Optical Module for HR and SpO₂ Measurement. Persons of skill in the art will appreciate that other integrated circuits may be employed without departing from the scope of the present subject matter and the present invention is not limited to the MAXM86161 Integrated Optical Module.

Different ways to measure SpO₂ may be applied for measurement. For example, SpO₂ measurement requires only two wavelengths. In various systems red and infrared wavelengths (typically 660 nm and 940 nm) are used. In various embodiments, other infrared wavelengths may be used across the range, such as wavelengths between 350 nm and 1200 nm. In various embodiments, three wavelengths are employed, such as red, green, and infrared. Other wavelengths and combinations may be used without departing from the scope of the present subject matter.

In the example of FIG. 2, the various ports of the integrated sensor 210 include, but are not limited to:
a. Port 202, which comprises a USB Type C Connector port (for low level charging of the computing device, such as a tablet computer, and for the wired connection to the tablet computer);
b. Port 204, which is a power supply port for the integrated sensor;
c. Port 206, which is an eight channel USB connector port with 4/8 BNC Analog Outputs;
d. Port 208, which is a four-pole connector port (which may be used to connect a pressure adapter and catheter);
e. Port 212, a four-pole stereo jack port (which may be used for external ECG needle electrodes)
f. Port 214, which is a connection for an optional pulse oximeter/SpO2 clip that may be used in conjunction with the optical sensors of the present disclosure; and
g. Thermocouple Probe Connector Port 216, which may be used to measure temperature of the subject animal, such as rectal temperature.

In various embodiments, the integrated sensor may include sense electronics for sensing one or more of ECG electrodes, temperature sensors, optical sensors, external SpO2 sensors, thermocouples, or other sensors. In various embodiments the integrated sensor may also include one or more of circuits for the Sp02 sensor (such as, but not limited to, the MAXM86161 Integrated Optical Module), heating electronics to apply heat and control applied heat, a heating element or other heating device, power supply electronics, electronics for sensing wired connections, such as a USB interface, wireless communications electronics and antennas, memory, and/or a processor, microprocessor, and/or digital signal processor, among other components.

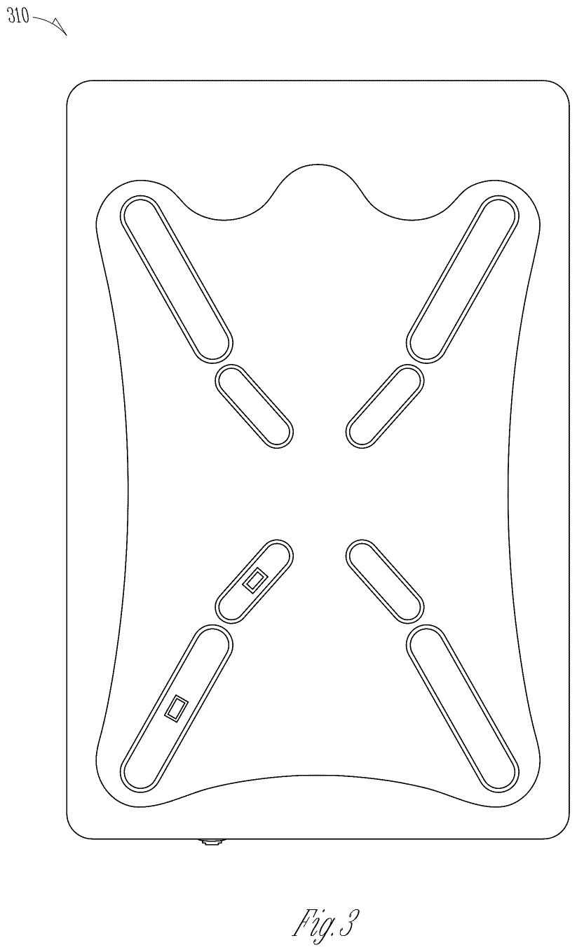

In various embodiments, the integrated sensor 210 includes an optional mounting bracket 218 to which an anesthetic nose cone may be attached to keep the subject animal sedated during testing and for other measurements. FIG. 3 shows an example profile of an integrated sensor 310 without an anesthesia mounting bracket 218 according to one embodiment of the present subject matter.

It is understood that combinations of ECG and Plethysmograph signals can be used to extract additional cardiac parameters such as pulse transit time (the time from QRS wave peak to onset of the plethysmograph blood flow signal). Such measurements are important as an increase in pulse transit time is associated with arterial stiffness. Pulse transit time can be varied by pharmaceutical drug interactions, which may be used in animal studies. For example, different drugs can be tested. Vasodilators will relax blood vessels and increase pulse transit time and vasoconstrictors will stiffen blood vessels and decrease pulse transit time.

In various embodiments, other blood parameters and wavelengths can be used to measure carboxyhemoglobin, lactate, troponin, among others. It is also possible to modify the electrode, optics, and electronics for fluorescence measurements. In various embodiments lasers can be used in the photoelectrode to allow for blood perfusion measurements.

In various embodiments photoacoustic measurements, ultrasonic doppler blood velocity measurements, and blood perfusion measurements (laser doppler) may be performed by augmenting the photoelectrode with acoustic transducers. Other measurements and sensors may be integrated without departing from the scope of the present subject matter.

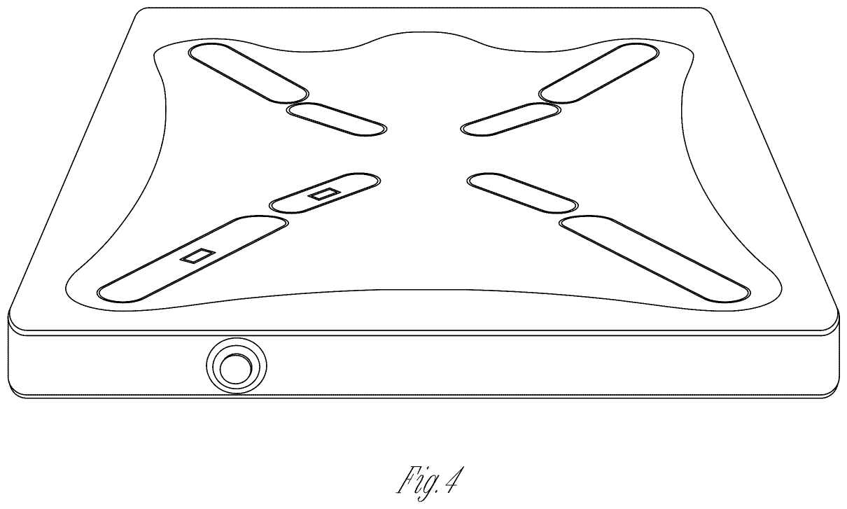

FIG. 4 is an end perspective view of the integrated sensor 310 of FIG. 3. The button 412 is a power button which may include a power indicator light in various embodiments. In various embodiments the power indicator light has additional functions. For example, the light may be an LED (light emitting diode) which lights up and blinks rapidly when the integrated sensor is powered up. In wireless embodiments, when a wireless communication is established with the computing device, the LED light stays solid on. In various embodiments, when the computing device is no longer running software to run the integrated sensor, the LED may blink rapidly and functions may be disabled, such as a the heater turning off, or other sensing functions may be disabled. The integrated sensor may be programmed so that the light will re-activate when software on the computing device is again executed to provide communications and control by the computing device of the integrated sensor. Those of skill in the art will appreciate that other control and signaling approaches may be employed without departing from the scope of the present subject matter.

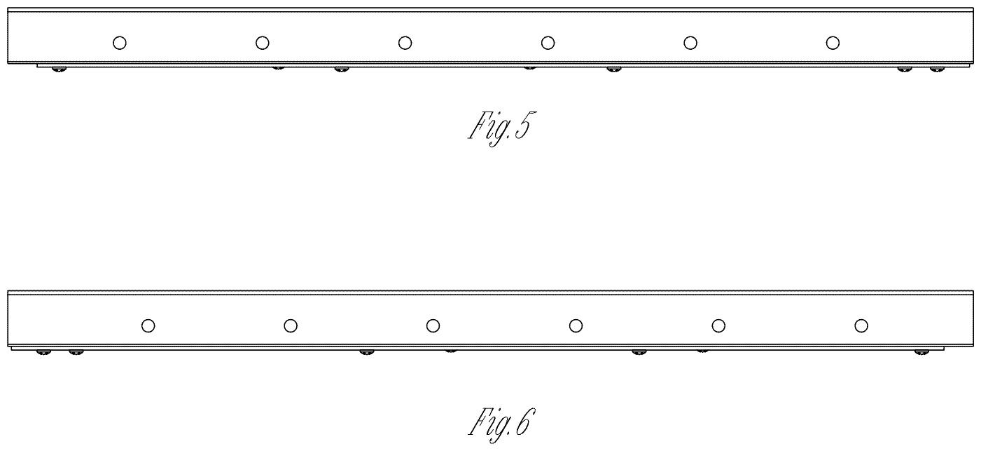

FIGS. 5 and 6 are right and left side views of the integrated sensor 310 of FIG. 3.

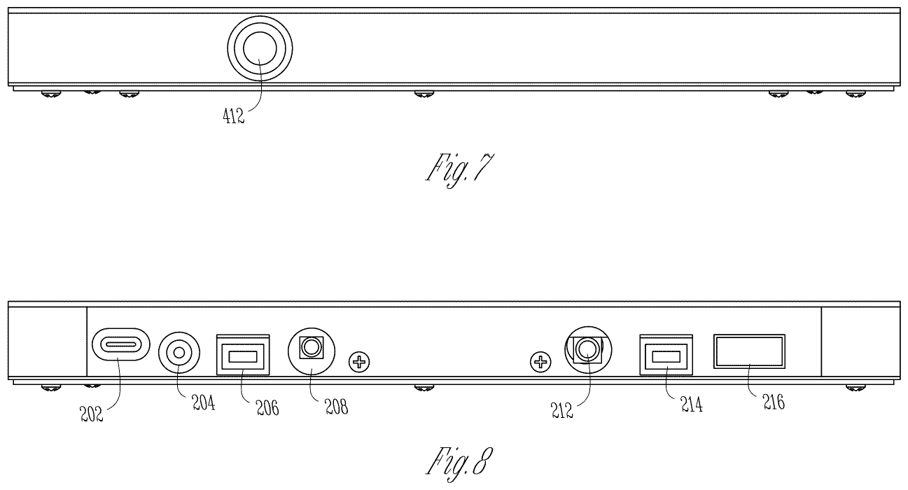

FIGS. 7 and 8 are bottom and top views of the integrated sensor 310 of FIG. 3, showing the power button from FIG. 4 and the ports previously described in FIG. 2.

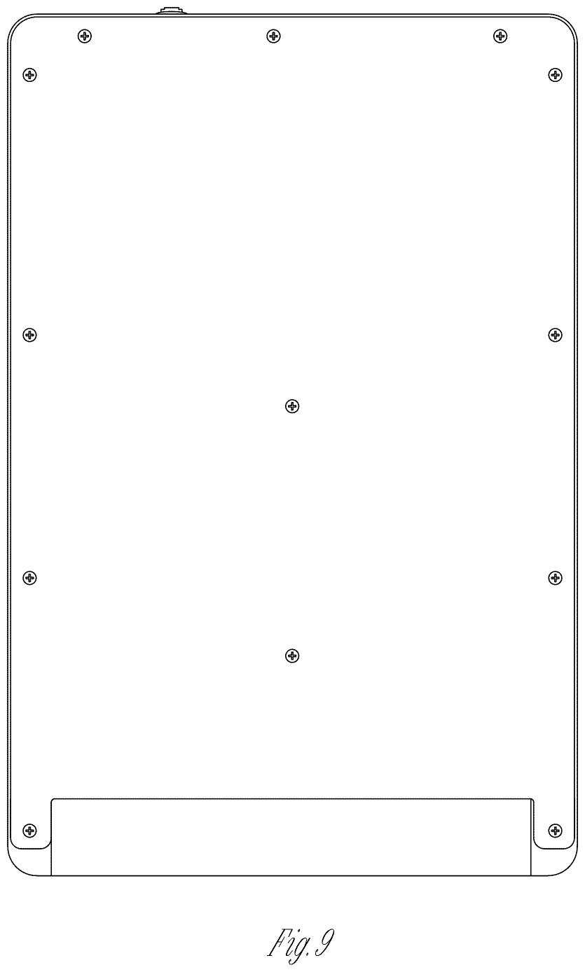

FIG. 9 is a back side view of the integrated sensor 310 of FIG. 3.

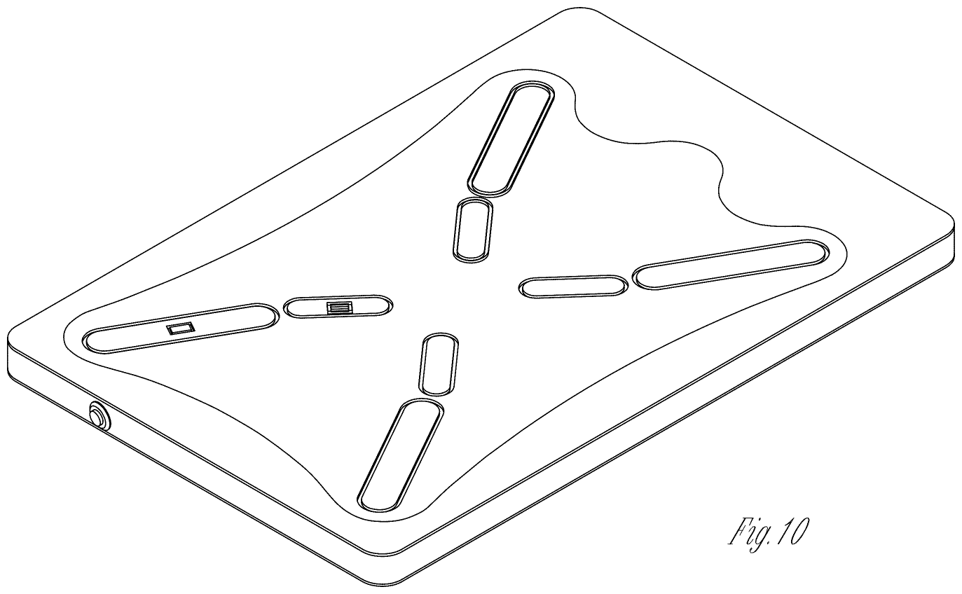

FIG. 10 is a side perspective view of the integrated sensor 310 of FIG. 3.

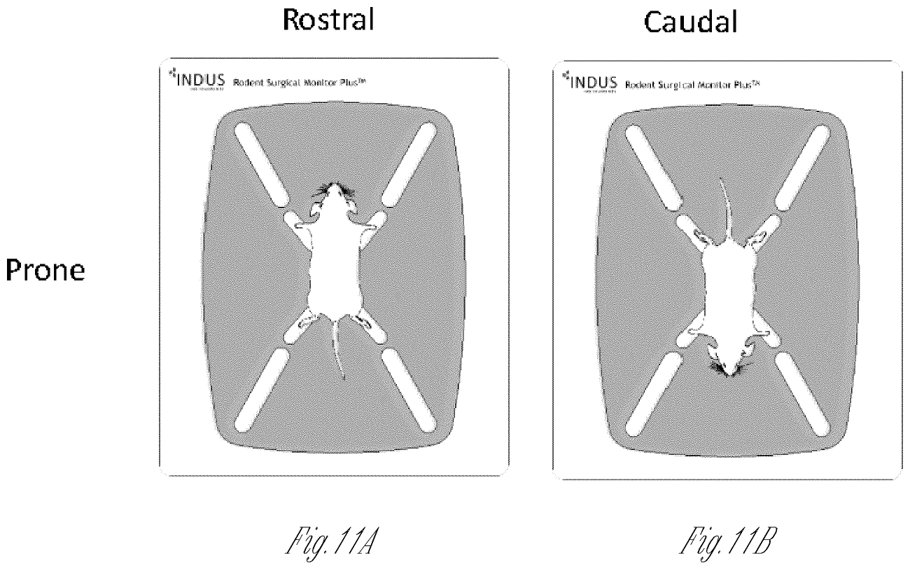

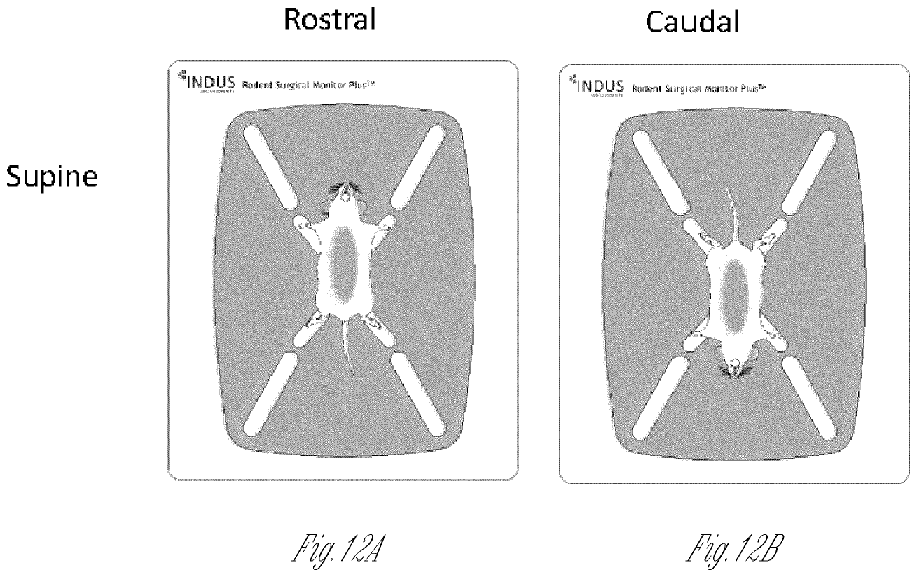

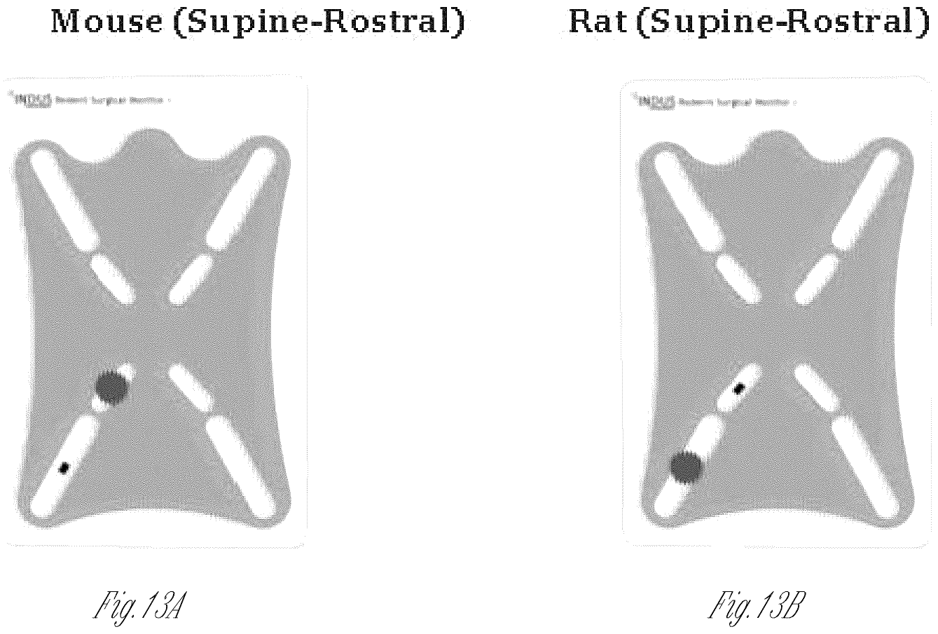

The integrated sensor may be used in several configurations for sensing electrophysiological parameters of the rodent subject animal, which involves positioning of the rodent paws on the electrodes in the manner shown in FIGS. 11A, 11B, 12A, and 12B. For example, FIGS. 11A and 11B demonstrate prone animal positioning on the sensor in the rostral (head towards the top of the integrated sensor) and caudal (tail towards the top of the integrated sensor) positions, respectively, according to various embodiments of the present subject matter. FIGS. 12A and 12B demonstrate supine animal positioning on the sensor in the rostral and caudal positions, respectively, according to various embodiments of the present subject matter. In various embodiments, as demonstrated in FIGS. 13A and 13B, the integrated sensor includes a paw pulse oximeter available on one electrode for the mouse or rat configuration designed to work in the Supine-Rostral position. Sensors can be implemented on other electrodes as well to allow for paw pulse oximeter to work in any of the positions shown above, and the present subject matter is not limited to these pad locations, pad shapes, or combinations.

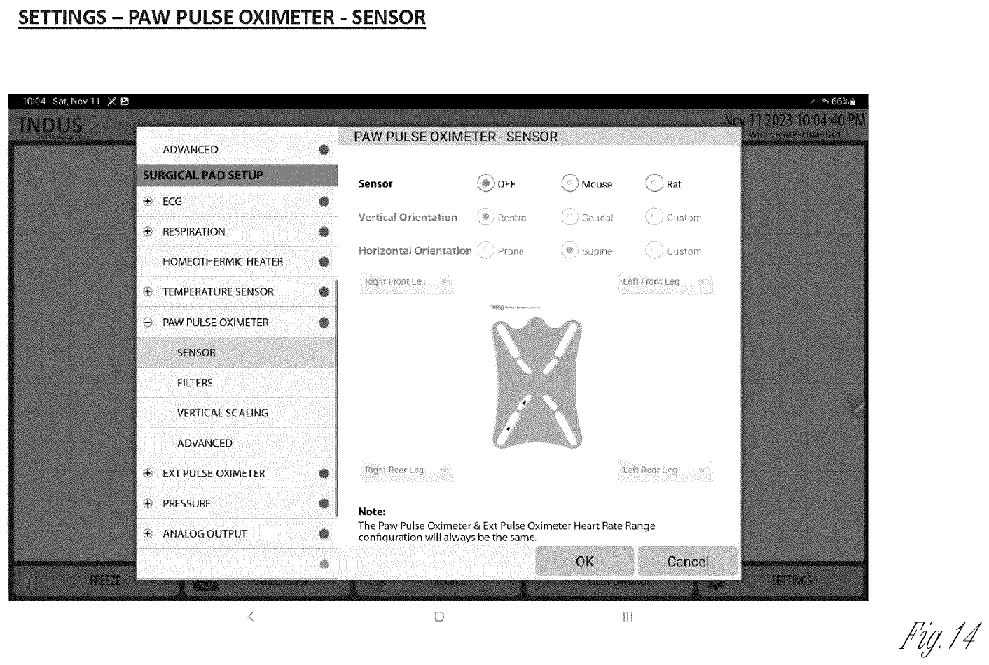

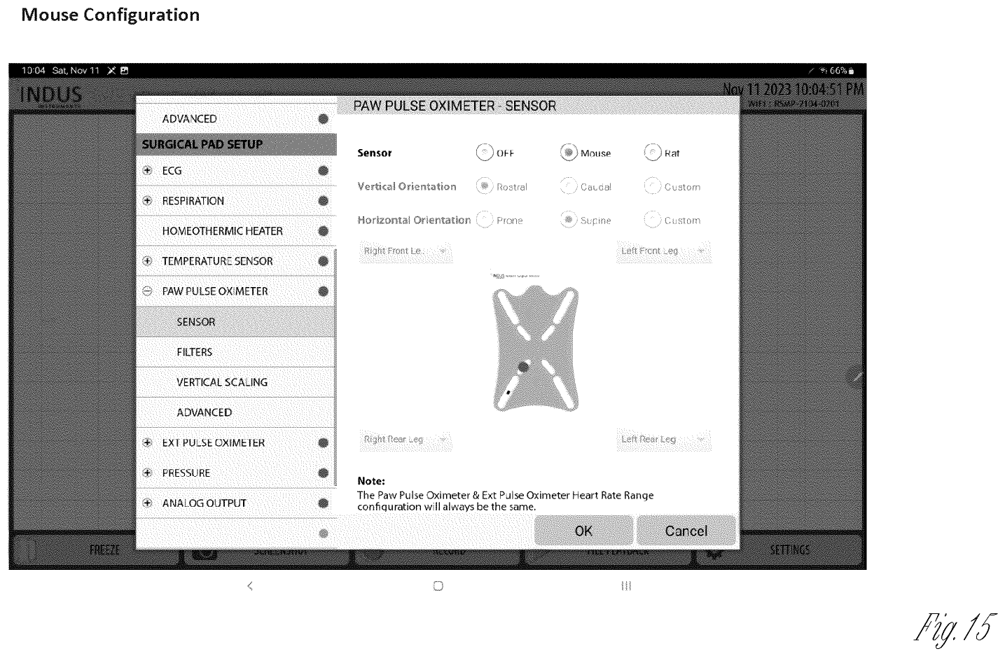

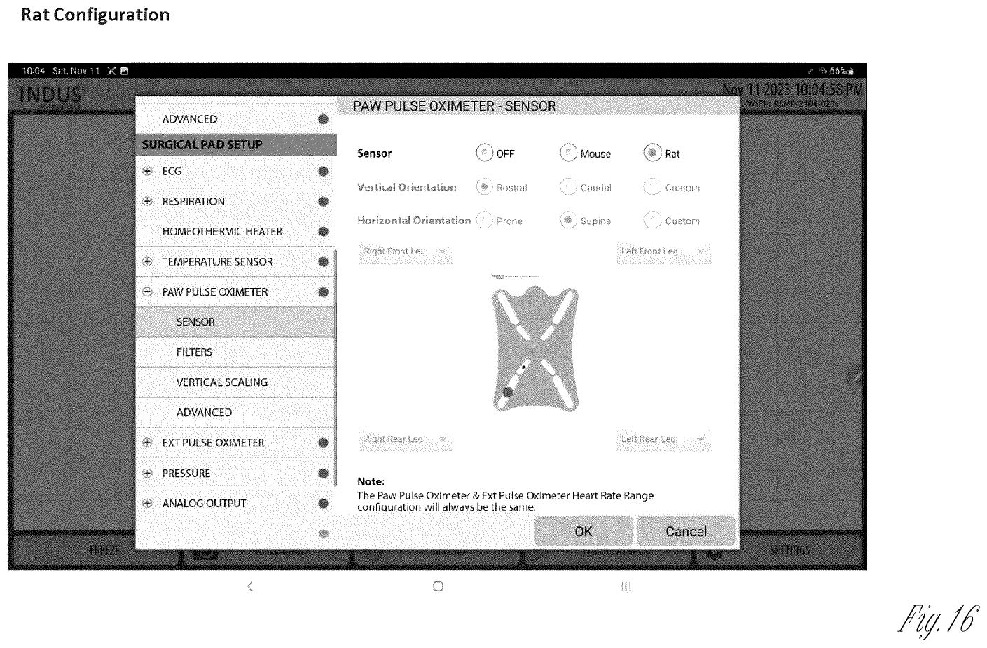

FIG. 14 is an example software interface for programming the pulse oximeter sensor, according to one embodiment of the present subject matter. The Paw Pulse Oximeter Sensor Type can be set to OFF (as shown in FIG. 14), Mouse or Rat (as shown in FIGS. 15-16) configurations. Unlike approaches of others, in the present application there is no need to remove the hair on the backside of the paw/foot of the subject animal. An electrode cream may be applied which does not interfere with red/infrared light transmission or reception of the LED sensors for pulse oximetry. The paw of the mouse/rat can be taped on to the electrode as long as the center of the paw is over the pulse oximeter sensor. It is not required to cover the foot/sensor area with a dark cloth but doing so may help minimize any potential interference from ambient light.

A heart rate range may be selected by the user of the interface that encompasses the expected heart rate of the animal. The Heart Rate Range (BPM) can be set to 240 to 960 (for mouse), 120 to 480 (for rat), 60 to 240 (naked mole-rats), or 30 to 120 (other small animals). Other rates, applications and settings are possible without departing from the scope of the present subject matter and the rodent examples are provided to demonstrate only one kind of sensing application.

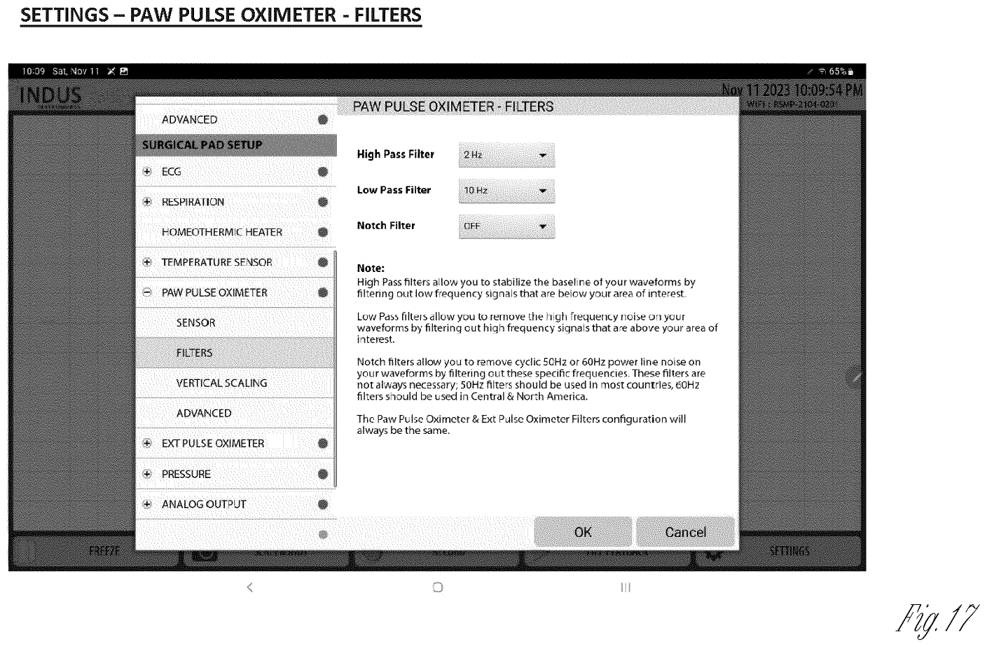

FIG. 17 shows an example of programming filters for the pulse oximetry sensor, according to one embodiment of the present subject matter. The high pass filter stabilizes the plethysmograph waveform baseline. The low pass filter removes high frequency noise that is superimposed on the plethysmograph waveform. The notch filter suppresses cyclic 50 Hz or 60 Hz AC power line noise that can interfere with the instrumentation.

As shown in the example interface, the high pass filter can be set to OFF, 0.2 Hz, 0.5 Hz, 1 Hz, 2 Hz, 5 Hz, or 10 Hz. The low pass filter can be set to OFF, 1 Hz, 2 Hz, 5 Hz, 10 Hz, 20 Hz, or 50 Hz. The notch filter can be set to OFF, 50 Hz, or 60 Hz.

In various embodiments, the software can be programmed to provide defaults that provide convenient starting places for users. For example, in one embodiment the software defaults to 2 Hz for the high pass filter, 10 Hz for the low pass filter, and “OFF” for the notch filter settings. Those of skill in the art will appreciate that other settings, parameters, and defaults are possible without departing from the scope of the present subject matter.

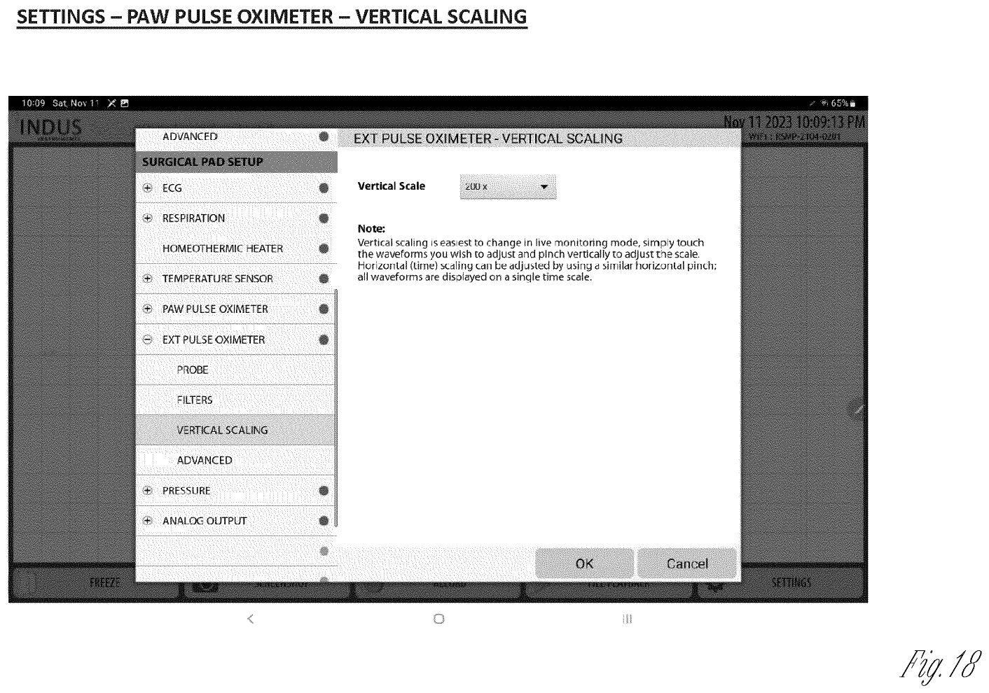

FIG. 18 shows an example of programming vertical scaling for the pulse oximetry sensor, according to one embodiment of the present subject matter. The vertical scale sets the number of scaled units per chart division. Vertical scaling has an inverse relationship to waveform amplitude; therefore, if the scaling is set high, the waveform will appear smaller. As shown in the example interface, the vertical scale can be adjusted to display 1×, 2×, 5×, 10×, 20×, 50×, 100×, 200×, 500×, 1000× units.

In various embodiments, the software can be programmed to provide vertical scaling defaults. For example, in one embodiment the software defaults to 200× for the vertical scale. Those of skill in the art will appreciate that other settings, parameters, and defaults are possible without departing from the scope of the present subject matter.

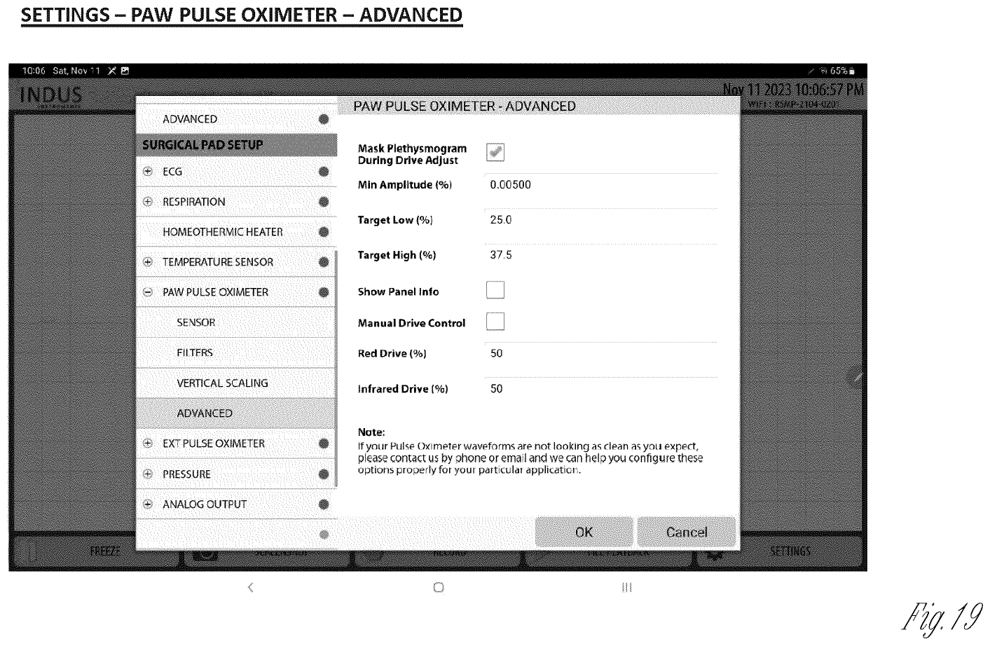

FIG. 19 shows an example of programming advanced features for the pulse oximetry sensor, according to one embodiment of the present subject matter. For example, one feature is “Mask Plethysmogram During Drive Adjust.” A user may check the box to enable the plethysmogram waveforms to be zeroed while an automatic control algorithm is actively adjusting the brightness of the LEDS (light emitting diodes) used to take the plethysmograph. The algorithm stops adjusting the LEDS when the DC levels of the plethysmogram waveforms are within the target zone (between “Target Low (%)” and “Target High (%)”; 0% to 100% is the fullscale range of the A/D converter).

The algorithm in the integrated sensor attempts to compute the SpO₂ only if the AC amplitudes of the plethysmogram waveforms are larger than “Min Amplitude (%)”;. 0% to 100% is the fullscale range of the A/D converter.

The “Show Panel Info” feature shows the result of the Red/IR ratio computation as well as the drive of the LEDs (0% to 100%); (displayed on the numeric panel under the SpO₂ number. This information can be used for debugging purposes.

The “Manual Drive Control” feature is used to manually set value for “Red Drive (%)” and “Infrared Drive (%).” When the “Manual Drive Control” box is checked the values entered for “Red Drive (%)” and “Infrared Drive (%)” take effect.

The calibration equation provided by the pulse oximeter sensor manufacturer is typically provided and generated for human use. The present subject matter adapts the signal processing to accommodate high signal frequencies (due to high heart rates) in mice & rats. Those of skill in the art will appreciate that other rates can be programmed for other applications without departing from the scope of the present subject matter.

The generated paw pulse plethysmograph red and infrared waveforms can be exported to be digitized and stored by external analog-to-digital acquisition systems.

### Signal Processing System

The system utilizes specialized algorithms to process the raw signals from the ECG electrodes and pulse oximetry sensor in order to calculate the key physiological parameters.

For the ECG data, it performs filtering, amplification, and R-wave detection to determine heart rate and heart rate variability. Abnormal cardiac rhythms and morphologies can also be detected through additional analysis.

The pulse oximetry waveforms undergo calibration and filtering to remove noise, adjust for optical properties of rodent blood, and isolate the pulsatile signals. Mathematical manipulation of the red and infrared signals generates saturations through empirically derived calibration equations. The pulse rate is also extracted from these waveforms.

The respiration rate is determined through filtering and processing of small modulations in the ECG and pulse oximetry signals that arise from chest expansion and contraction.

Advanced algorithms are also incorporated to provide surrogate blood pressure measurements derived from subtle variations in the morphologies of beats and pulses over time.

These specialized signal analysis techniques allow accurate determination of the key parameters from rodents and other small animals despite the challenges of their size and physiology. The algorithms are tuned to extract the maximum useful data from the sensors.

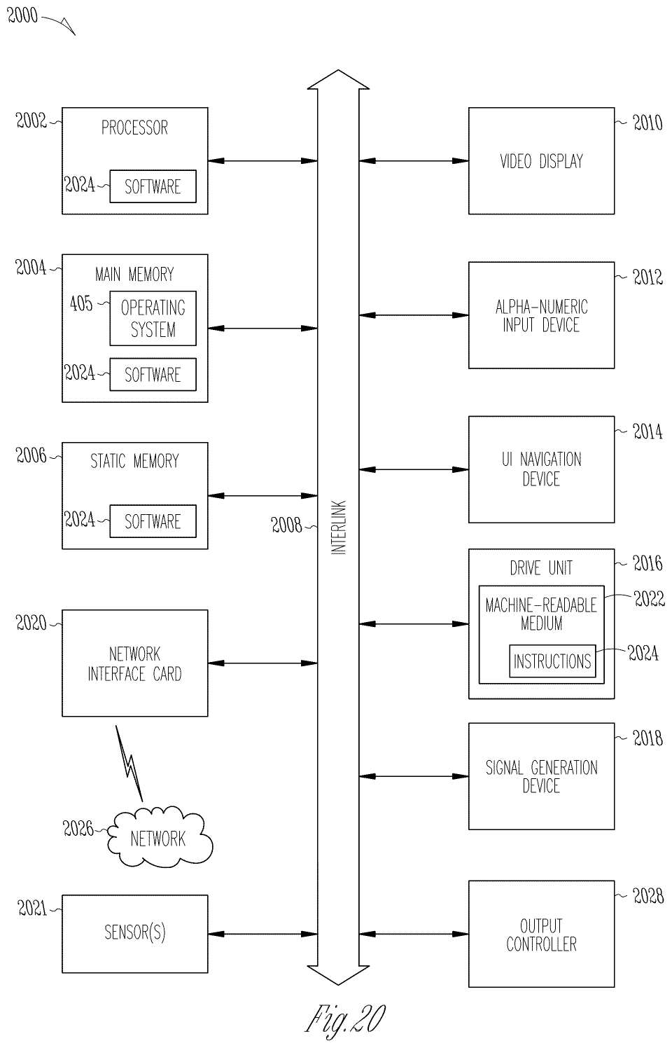

FIG. 20 is a block diagram of a computing device in the example form of a computing system within which a set of instructions may be executed, for causing the machine to perform any one or more of the methodologies discussed herein.

These different components can be located on a single device, multiple devices in one location, or multiple objects in various locations. A network may be used to interconnect any two or more of these modules. In the case of remote devices, the network may be a local area network (LAN), the INTERNET, a personal area network, a wireless network, other networks, or any combination of these networks.

FIG. 20 illustrates a block diagram of an example machine 2000 upon which any one or more of the techniques (e.g., methodologies) discussed herein may perform. In alternative embodiments, the machine 2000 may operate as a standalone device or may be connected (e.g., networked) to other machines. In a networked deployment, the machine 2000 may operate in the capacity of a server machine, a client machine, or both in server-client network environments. In an example, the machine 2000 may act as a peer machine in peer-to-peer (P2P) (or other distributed) network environment. The machine 2000 may be in the form of a personal computer (PC), a tablet PC, a set-top box (STB), a personal digital assistant (PDA), a mobile telephone, a smart phone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

Machine (e.g., computer system) 2000 may include a hardware processor 2002 (e.g., a central processing unit (CPU), a graphics processing unit (GPU), a hardware processor core, or any combination thereof), a controller, a microcontroller, a microprocessor, a main memory 2004 and a static memory 2006, some or all of which may communicate with each other via an interlink (e.g., bus) 2008. The machine 2000 may further include a display unit 2010, an alphanumeric input device 2012 (e.g., a keyboard), and a user interface (UI) navigation device 2014 (e.g., a mouse). In an example, the display unit 2010, input device 2012 and UI navigation device 2014 may be a touch screen display. The machine 2000 may additionally include a storage device (e.g., drive unit) 2016, a signal generation device 2018 (e.g., a speaker), a network interface device 2020, and one or more sensors 2021, such as a global positioning system (GPS) sensor, compass, accelerometer, or other sensor. The machine 2000 may include an output controller 2028, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 2016 may include a machine readable medium 2022 on which is stored one or more sets of data structures or instructions 2024 (e.g., software) embodying or utilized by any one or more of the techniques or functions described herein. The instructions 2024 may also reside, completely or at least partially, within the main memory 2004, within static memory 2006, or within the hardware processor 2002 during execution thereof by the machine 2000. In an example, one or any combination of the hardware processor 2002, the main memory 2004, the static memory 2006, or the storage device 2016 may constitute machine readable media.

While the machine readable medium 2022 is illustrated as a single medium, the term “machine readable medium” may include a single medium or multiple media (e.g., a centralized or distributed database, and/or associated caches and servers) configured to store the one or more instructions 2024.

The term “machine readable medium” may include any medium that is capable of storing, encoding, or carrying instructions for execution by the machine 2000 and that cause the machine 2000 to perform any one or more of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); Solid State Drives (SSD); and CD-ROM and DVD-ROM disks. In some examples, machine readable media may include non-transitory machine-readable media. In some examples, machine readable media may include machine readable media that is not a transitory propagating signal.

The instructions 2024 may further be transmitted or received over a communications network 2026 using a transmission medium via the network interface device 2020. Machine 2000 may communicate with one or more other machines utilizing any one of a number of transfer protocols (e.g., frame relay, internet protocol (IP), transmission control protocol (TCP), user datagram protocol (UDP), hypertext transfer protocol (HTTP), etc.). Example communication networks may include wired and wireless communications, such as Ethernet, Bluetooth, Bluetooth Low Energy, other Personal Area Networks (PANs), LoRa, NFC, Wi-Fi, WiMAX, 3G, 4G, LTE, 5G, the unlicensed 915 MHz Industrial, Scientific, and Medical (ISM) frequency band, Zigbee, among others. Some standards may support mesh networks. The networks include, but are not limited to, a local area network (LAN), a low-power wide-area network (LPWAN), a wide area network (WAN), a packet data network (e.g., the Internet), mobile telephone networks (e.g., cellular networks), Plain Old Telephone (POTS) networks, and wireless data networks, e.g., Institute of Electrical and Electronics Engineers (IEEE) 802.11 family of standards known as Wi-Fi®, IEEE 802.16 family of standards known as WiMax®, NFC, IEEE 802.15.4 family of standards, a Long Term Evolution (LTE) family of standards, a Universal Mobile Telecommunications System (UMTS) family of standards, peer-to-peer (P2P) networks, among others. The NFC circuitry may be embodied as relatively short-range, high frequency wireless communication circuitry and may implement standards such as ECMA-340/ISO/IEC 18092 and/or ECMA-352/ISO/IEC 21481 to communicate with other devices. In an example, the network interface device 2020 may include one or more physical jacks (e.g., Ethernet, coaxial, or phone jacks) or one or more antennas to connect to the communications network 2026. In an example, the network interface device 2020 may include a plurality of antennas to wirelessly communicate using at least one of single-input multiple-output (SIMO), multiple-input multiple-output (MIMO), or multiple-input single-output (MISO) techniques. In some examples, the network interface device 2020 may wirelessly communicate using Multiple User MIMO techniques.

The foregoing examples are not intended to be an exhaustive or exclusive list of examples and variations of the present subject matter. The above description is intended to be illustrative, and not restrictive. Those of skill in the art will appreciate additional variations of the embodiments that can be used within the scope of the teachings set forth herein. The scope of the invention should be determined with reference to the appended claims, along with the full scope of equivalents to which such claims are entitled.

What is claimed is:

1. An integrated sensor for physiological measurements of a subject animal and communications with a computing device, the integrated sensor comprising:
at least one set of electrodes configured to sense at least one electrophysiological parameter of the subject animal placed on the electrodes, the electrodes each having an outline;
at least one optical sensor disposed in the outline of at least one of the electrodes, the optical sensor for plethysmographic measurements;
communication electronics to provide communications with the computing device.

2. The integrated sensor of claim 1, wherein the electrodes are configured to measure electrocardiogram (ECG) data from the subject animal.

3. The integrated sensor of claim 1, wherein the optical sensor is configured to measure pulse oximetry (SpO2) data from the subject animal.

4. The integrated sensor of claim 1, further comprising a heating element configured to provide adjustable levels of heat to the subject animal.

5. The integrated sensor of claim 1, wherein the communication electronics are configured to communicate wirelessly with the computing device.

6. The integrated sensor of claim 1, wherein the communication electronics are configured to communicate with the computing device via at least one of WiFi, Bluetooth, Zigbee, LoRa, NFC, WiMAX, 3G, 4G, 5G, LTE, or 915 MHz ISM frequency band.

7. The integrated sensor of claim 1, further comprising a temperature probe connector port for measuring the temperature of the subject animal.

8. The integrated sensor of claim 1, wherein the at least one optical sensor is further configured to be used without the need for hair removal on the subject animal.

9. The integrated sensor of claim 1, wherein the at least one set of electrodes includes separate configurations for different types of subject animals.

10. The integrated sensor of claim 1, wherein the at least one set of electrodes is configured to facilitate the placement of the subject animal in both prone and supine positions.

11. The integrated sensor of claim 1, wherein the at least one optical sensor includes a special purpose integrated circuit for SpO2 measurement.

12. The integrated sensor of claim 1, wherein the at least one optical sensor is disposed in a predefined location on the electrode to optimize sensing based on the anatomy of the subject animal.

13. The integrated sensor of claim 1, further comprising a software interface for programming the pulse oximeter sensor, the interface allowing selection of heart rate ranges and filter settings.

14. The integrated sensor of claim 1, wherein the integrated sensor is part of a system that includes a computing device with a display for real-time and post-processed data sensed from the subject animal.

15. The integrated sensor of claim 1, wherein the integrated sensor is configured to process high-frequency signals from small animals to provide accurate measurements of physiological parameters.

16. A non-transitory computer-readable medium having stored thereon instructions that, when executed by a processor of a computing device, cause the computing device to perform operations comprising:
receiving data from an integrated sensor configured for physiological measurements of a subject animal, the integrated sensor including at least one set of electrodes and at least one optical sensor;
processing the received data to determine one or more physiological parameters of the subject animal, wherein the physiological parameters include at least electrocardiogram (ECG) data and pulse oximetry (SpO2) data;
applying a signal processing algorithm adapted for high-frequency signals from small animals to enhance the accuracy of the physiological parameters;
displaying the physiological parameters on a user interface of the computing device;
enabling user interaction with the user interface to configure settings of the integrated sensor, including selection of heart rate ranges, filter settings, and sensor calibration; and
wirelessly transmitting control signals to the integrated sensor to adjust operational parameters based on the user-configured settings.

17. The non-transitory computer-readable medium of claim 16, wherein the signal processing algorithm includes filtering techniques to remove noise from the ECG and SpO2 data.

18. The non-transitory computer-readable medium of claim 16, wherein the user interface provides real-time visualization of the physiological parameters through graphical representations.

19. The non-transitory computer-readable medium of claim 16, wherein the user-configured settings include the ability to select between different animal profiles to optimize the integrated sensor for different species.

20. The non-transitory computer-readable medium of claim 16, wherein the operations further comprise exporting the processed physiological parameters to an external data storage or analysis system.

21. The non-transitory computer-readable medium of claim 16, wherein the operations further comprise generating alerts based on the detection of abnormal physiological parameters.

22. The non-transitory computer-readable medium of claim 16, wherein the operations further comprise calibrating the integrated sensor based on a set of calibration parameters entered through the user interface.

23. The non-transitory computer-readable medium of claim 16, wherein the operations further comprise adjusting the level of heat provided by a heating element of the integrated sensor to maintain the subject animal's body temperature.

24. The non-transitory computer-readable medium of claim 16, wherein the operations further comprise recording and storing a history of the physiological parameters for subsequent analysis.

25. The non-transitory computer-readable medium of claim 16, wherein the operations further comprise enabling the user to manually override automatic settings of the integrated sensor through the user interface.

26. The non-transitory computer-readable medium of claim 16, wherein the operations further comprise providing a tutorial or guidance feature on the user interface to assist the user in positioning the subject animal on the integrated sensor.
